(19) United States
(12) Patent Application Publication
(10) Pub. No.: US 2018/0125697 A1
(43) Pub. Date: May 10, 2018

(54) Valve Assembly for Modifying Flow in a Catheter System

(71) Applicant: Mehana Medical LLC, Coto De Caza, CA (US)

(72) Inventor: David Ferrera, Coto De Caza, CA (US)

(21) Appl. No.: 15/573,453

(22) PCT Filed: May 10, 2016
(86) PCT No.: PCT/US16/31650

§ 371 Date: Nov. 10, 2017

Related U.S. Application Data
(60) Provisional application No. 62/159,740, filed on May 11, 2015.

(51) Int. Cl. A61F 5/44; A61M 39/24

(52) U.S. Cl. CPC A61F 5/4405; A61M 25/0017; A61M 39/24

(57) ABSTRACT

The present invention relates to fluid flow diversion in medical procedures, and, more particularly, to a valve assembly for preventing retrograde flow of urine from a collection device into a urinary catheter so as to reduce the risk of and/or prevent catheter-associated urinary tract infection (CAUTI). The valve assembly of the present invention is a discrete, stand-alone valve assembly configured to provide a fluid connection between a urinary catheter (e.g., Foley catheter), and a collection device (e.g., urine drainage bag) or the tubing for coupling the catheter to the collection device. The valve assembly is configured to permit antegrade flow of urine from the catheter into the collection device, while preventing retrograde flow of urine (e.g., backflow) from the collection device into the catheter, thereby reducing the risk of CAUTI.

### BACKGROUND OF THE INVENTION

It is critical to one's health to maintain an uninterrupted flow of urine from the body. If urinary flow is interrupted, either completely or partially, a person may suffer serious complications, which can be fatal. It is very common for patients, particularly in health care facilities, to require a urinary diversion of one form or another. One type of urinary diversion includes urinary catheterization, a procedure involving the insertion of a drainage catheter, such as a Foley catheter, into a patients bladder via the urethra. Catheterization allows the patient's urine to drain freely from the bladder for subsequent collection, wherein the drainage catheter may be coupled to an external urine collection system, such as a drainage bag or container, so as to allow monitoring of urine output. During long-term use, the catheter may be left in place all the time, or a patient may be instructed on a procedure for placing a catheter just long enough to empty the bladder and then removing if (known as intermittent self-catheterization). Patients undergoing major surgery are often catheterized and may remain so for some time.

Hospitals, and other medical facilities providing patient care, are coming under greater pressure to reduce iatrogenic complications. One common complication that is undergoing increasing scrutiny is the hospital acquired urinary catheter infection (UTI) or a catheter-associated urinary tract infection (CAUTI). Despite the implementation of cautionary measures for preventing UTI's, including important hygienic measures for handling catheterization systems, the urinary tract may frequently become infected during a hospital stay due urine backflow. For example, the flow of urine through common urinary catheterization systems occurs under the influence of gravity. However, in some instances, collected urine, may flow back into the urinary tract (urine backflow), which is presently a major cause of CAUTI.

Urine backflow may occur for a variety of reasons, including maneuvering of the patient, which may result in the collection container/bag (filled with collected urine) being raised above the level of the bladder, thereby resulting in the reflux of urine back into the bladder via the catheter under the influence of gravity. While in the collection container/bag, the corrected urine may reside there for a period of time, at which point collected urine has a good chance to develop bacteria. Manipulation of the collection container/bag relative to the patients bladder may provide a pathway for bacteria-ridden urine from the collection container/back to travel back into the urinary tract and into the bladder, thereby causing infections. The most common symptoms of a UTI are burning sensation with urination, flank pain, sever or nausea and vomiting. If left untreated, a UTI may result in kidney infection and sepsis, possibly resulting in death.

### BRIEF SUMMARY OF THE INVENTION

The present invention relates to fluid flow diversion in medical procedures, and, more particularly, to a valve assembly for preventing retrograde flow of urine from a collection device into a urinary catheter so as to reduce the risk of and/or prevent catheter-associated urinary tract infection (CAUTI). The valve assembly of the present invention is a discrete, stand-atone device configured to provide a fluid connection between a urinary catheter (e.g., Foley catheter) and a collection device (e.g., urine drainage bag) or the tubing for coupling the catheter to the collection device. The valve assembly is configured to permit antegrade flow of urine from the catheter into the collection device, while preventing retrograde flow of urine (e.g., backflow) from the collection device into the catheter, thereby reducing the risk of CAUTI.

The valve assembly generally includes a housing having an inlet end configured to be directly connected to a drain port of the catheter and an outset end configured to be directly connected to an inlet port of the collection device or an Net end of the robing to be coupled to the collection device. The valve assembly further includes a valve positioned within the housing. The valve has an open inlet, an adjustable outlet and a fluid pathway extending there between. The open inlet of the valve is configured to receive fluid from the drain port, of the catheter and the adjustable outlet or the valve is configured to move from a normally closed position to an open position in response to receipt of fluid from the catheter so as to permit fluid to flow through the valve and valve assembly in an antegrade fashion. The adjustable outlet of the valve is further configured to return to the normally closed position (or remain in the normally closed position) in response to receipt of fluid from the collection device so as to prevent retrograde fluid flow (e.g., flow of fluid through the valve assembly and into the catheter).

The valve assembly of the present invention is a stand-alone device configured to be exchangeable and fit between most urinary catheters and collection bags and/or collection bag tubing. The one-way valve allows for easy and rapid passage of urine under minimal pressure (e.g., approximately 3 omH₂O), so as to allow the catheter to drain urine in a typical antegrade fashion, while efficiently preventing urine backflow by instantaneously closing with any movement of urine in a retrograde fashion. The valve assembly can be single use, disposable, and provided individually in a pre-sterilized package. Furthermore, housing of the valve assembly may be constructed of a transparent or translucent material, so as to provide a substantially unobstructed view of the valve so as to ensure proper orientation of the valve direction, as well as allow a clinician to ascertain the working condition of the valve (e.g., clogging or malfunction of the valve).

According to embodiments described herein, a valve assembly for preventing retrograde flow of fluid in a drainage catheter is provided. The valve assembly includes a housing having an inlet end and an outlet end. The inlet end is configured to be couplable to a drainage catheter. The valve assembly further includes a valve positioned within the housing and having an open inlet, an adjustable outlet, and a fluid pathway extending there between. The open inlet of the valve is configured to receive fluid from the drainage catheter and the adjustable outlet of the valve is configured to move between a closed position and an open position in response to receipt of fluid from the drainage catheter. When the adjustable outlet is in an open position, fluid is permitted to flow through the valve assembly in an antegrade direction from the inlet end towards the outlet end of the housing, thereby permitting flow of fluid from the drainage catheter. When the adjustable outlet is in a closed position, fluid is prevented from flowing through the valve assembly in a retrograde direction from the outlet end towards the inlet end of the housing, thereby preventing backflow of fluid into the drainage catheter.

In some embodiments, the adjustable outlet of the valve is configured to move from the closed position to the open position in response to receipt of fluid flow from the drainage catheter having a fluid pressure equal to or greater than a predefined threshold. The predefined threshold may be 3 cmH₂O pressure. The adjustable outlet of the valve may be configured to return to the closed position in response to a lack of fluid flow from the drainage catheter having a fluid pressure equal to or greater than a predefined threshold. The adjustable outlet of the valve is configured to move from the open position to the closed position in response to receipt of fluid from the outlet end of the housing.

In some embodiments, the outlet end of the housing is configured to be coupled to a collection device configured to receive and collect fluid draining from the drainage catheter. The collection device may include, but is not limited to, a container, a bag, or the like for collecting and storing fluid for a given period of time so as to allow monitoring of fluid flow, monitoring of the condition (e.g., appearance) of the fluid, as well as permitting sampling of the collected fluid for laboratory testing. The valve assembly is configured to prevent retrograde flow of fluid from the collection device into the drainage catheter.

In some embodiments, the valve comprises first and second elongated members joined along respective longitudinal edges thereof and sized to substantially span the valve inlet and valve outlet. The valve may be constructed of an elastomeric material.For example, the valve may be constructed from at least one of polyurethane, polyvinyl chloride, polyester, polyethylene, polyamide, silicone, polyisoprene, or a blend of at least two thereof. The housing of the valve assembly may be constructed of a transparent or translucent material configured to provide a view into an interior cavity of the housing.

According to another aspect, a drainage catheter system for draining fluid from s patient is generally provided. The system includes a catheter comprising an elongate tubular body having a proximal end and a distal end. The proximal end has a drainage opening and the distal end has at least a drainage port in fluid communication with the drainage opening via a drainage lumen extending along a length of the body from the side drainage opening to the drainage port. In some embodiments, the catheter is a urinary catheter configured to allow drainage of urine from a patient's bladder. The system further includes a collection device coupled to the catheter and configured to collect fluid received from the catheter. In some embodiments, the collection device is a urine collection bag, for example.

The system further includes a valve assembly disposed between the catheter and the collection device and provides a connection there between. The valve assembly includes a housing having an inlet end connected to the drainage port of the catheter and an outlet end connected to a portion of the collection device. The valve assembly further includes a valve positioned within the housing. The valve has an open inlet an adjustable outlet, and a fluid pathway extending there between. The open inlet or the valve is engaged with the housing inlet end and configured to receive fluid from the catheter. The adjustable outlet of the valve is configured to move between a normally closed position and an open position in response to receipt of fluid from the catheter.

When the adjustable outlet is in an open position, fluid is permitted to flow through the valve assembly in an antegrade direction from the inlet end towards the outlet end of the housing, so as to permit the flow of fluid from the catheter to the collection device. When the adjustable outlet is in a closed position, fluid is prevented from flowing through the valve assembly in a retrograde direction from the outlet end towards the inlet end of the housing, thereby preventing backflow of fluid from the collection device into the catheter.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein, it is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” used herein in the specification and in the claims, unless clearly indicated to the contrary, should be understood to mean “at least one.”

The phrase “and/or,” as used herein in the specification and in the claims, should be understood to mean “either or both” of the elements so con joined, i.e., elements that are conjunctively present n some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The forms and expressions which have been employed herein are used as terms, of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### BRIEF DESCRIPTION OF THE DRAWINGS

FIG. 1 is a perspective view of a Foley catheter shown in an uninflated state.

FIG. 2 is an enlarged view of the proximal end of the Foley catheter of FIG. 1 illustrating the balloon in an inflated state.

FIG. 3 is a perspective view of a catheter system including a valve assembly for modifying urinary flow.

FIG. 4 is an enlarged side view of the valve assembly of FIG. 3 consistent with the present disclosure.

FIG. 5A is a side view, partly in section, of the valve assembly of FIG. 4 illustrating the valve in an open position.

FIG. 58 is a side view, partly in section, of the valve assembly of FIG. 4 illustrating the valve in a normally closed position.

### DETAILED DESCRIPTION OF THE INVENTION

By way of overview, the present invention is generally directed to a valve assembly for preventing retrograde flow of urine from a collection device into a urinary catheter so as to reduce the risk of and/or prevent catheter-associated urinary tract infection (CAUTI). The valve assembly of the present invention is a discrete, stand-along valve assembly configured to provide a fluid connection between a urinary catheter (e.g., Foley catheter) and a collection device (e.g., urine drainage bag) or the tubing for coupling the catheter to the collection device. The valve assembly is configured to permit antegrade flow of urine from the catheter into the collection device, while preventing retrograde flow of urine (e.g., backflow) from the collection device into the catheter, thereby reducing the risk of CAUTI. FIG. 8 shows a fully assembled catheter system with valve according to embodiments of the present invention.

The valve assembly generally includes a housing having an inlet end configured to be directly connected to a drain port of the catheter and an outlet aid configured to: be directly connected to an inlet port of the collection device or an inlet end of the tubing to be coupled to the collection device. The valve assembly further includes a valve positioned within the housing. The valve has an open inlet, an adjustable outlet, and a fluid pathway extending there between. The open inlet of the valve is configured to receive fluid from the drain port of the catheter and the adjustable outlet of the valve is configured to move from a normally closed position to an open position in response to receipt of fluid from the catheter so as to permit fluid to flow through the valve and valve assembly in an antegrade fashion. The adjustable outlet of the valve is further configured to return to the normally closed position (or remain in the normally closed position) in response to receipt of fluid from the collection device so as to prevent, retrograde fluid flow (e.g., flow of fluid through the valve assembly and into the catheter).

The valve assembly of the present invention is a stand-alone device configured to be exchangeable and fit between most urinary catheters and collection hags and/or collection bag tubing. The one-way valve allows for easy and rapid passage of urine under minimal pressure (e.g., approximately 3 cmH₂O), so as to allow the catheter to drain urine in a typical antegrade fashion, while efficiently preventing urine backflow by instantaneously closing with any movement of urine in a retrograde fashion. The valve assembly can be single use, disposable, and provided individually in a pre-sterilized package. Furthermore, housing of the valve assembly may be constructed of a transparent or translucent material, so as to provide a substantially unobstructed view of the valve so as to ensure proper orientation of the valve direction, as well as allow a clinician to ascertain the working condition of the valve (e.g., dogging or malfunction of the valve).

According to embodiments described herein, a valve assembly consistent with the present disclosure is configured to be used with a drainage catheter and a collection device configured to collect fluid drained from the drainage catheter. For example, in some embodiments described herein, the valve assembly is configured to couple a urinary catheter to a collection device, such as a urine collection bag. However, it should be noted that the valve assembly of the present disclosure can be used with any type of catheter through which fluid is to drain out of the patient, and is not be limited solely to use with a urinary catheter. For example, the valve assembly may be used with catheters used for draining fluid from other organs/cavities/systems of the human body.

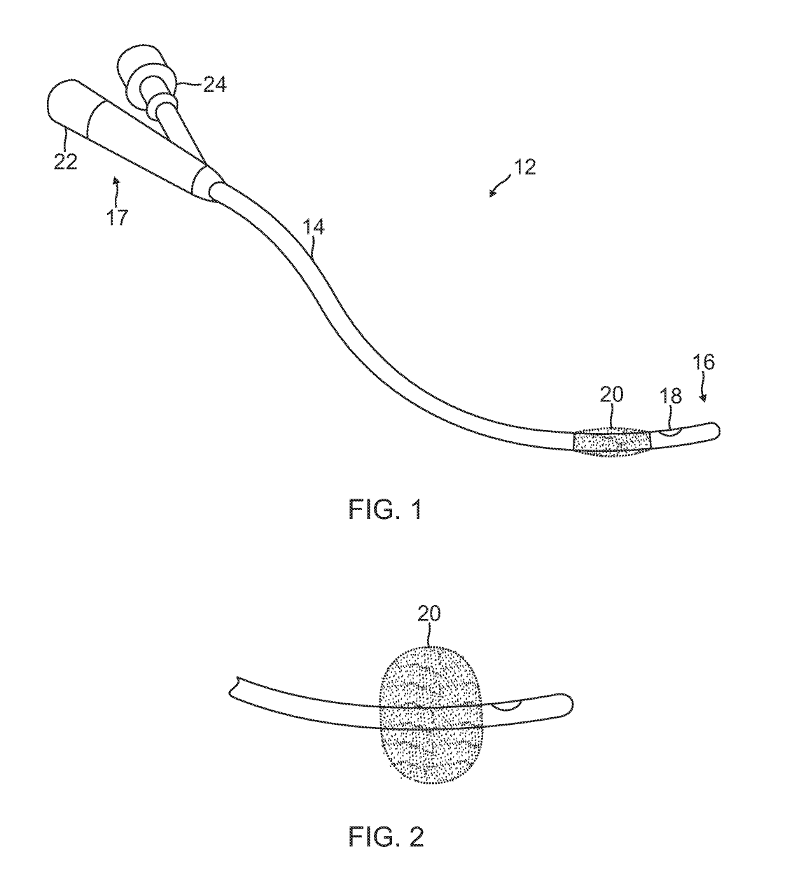

The urinary catheter may include a Foley catheter, as shown in FIG. 1. In the illustrated embodiment, the common Foley catheter 12 may generally include an elongate tubular body 14 having a proximal end 16 and a distal end 17. The proximal end 16 is configured to be inserted into a patient's urethra and advanced into the bladder. The proximal end 16 includes a side drainage opening 18 configured to receive urine from within the bladder. The proximal end 16 further includes a balloon 20 configured to be inflated while positioned within the bladder so as to prevent the proximal end 16 from withdrawing from the bladder, thereby maintaining positioning of the side drainage opening 18 to receive urine. As shown in FIG. 25 the balloon is in an inflated state.

It should be noted that the valve assembly of the present disclosure may be used with other types of urinary catheters, and is not limited to use with a Foley catheter, or other type of indwelling catheter having a balloon. For example, the valve assembly may be used with balloon-free urinary catheters, such as, for example, condom catheters, intermittent urinary catheters (e.g., Robinson catheter), and the like.

The distal end 17 includes at least a drainage port 22 in fluid communication with the drainage opening 18 via a drainage lumen (not shown) extending along a length of the body 14 from the side drainage opening 18 to the drainage port 22. The distal end 17 further includes a balloon port 24 in fluid communication with the balloon 20 via an inflation lumen extending along a length of the body 14. A clinician can inflate the balloon 20 by introducing a fluid (e.g., sterile water) into the balloon port and effectively inflating the balloon 20. Once positioned within the bladder, urine can flow into the side drainage opening 18, through the drainage lumen, and out of the drainage port 22.

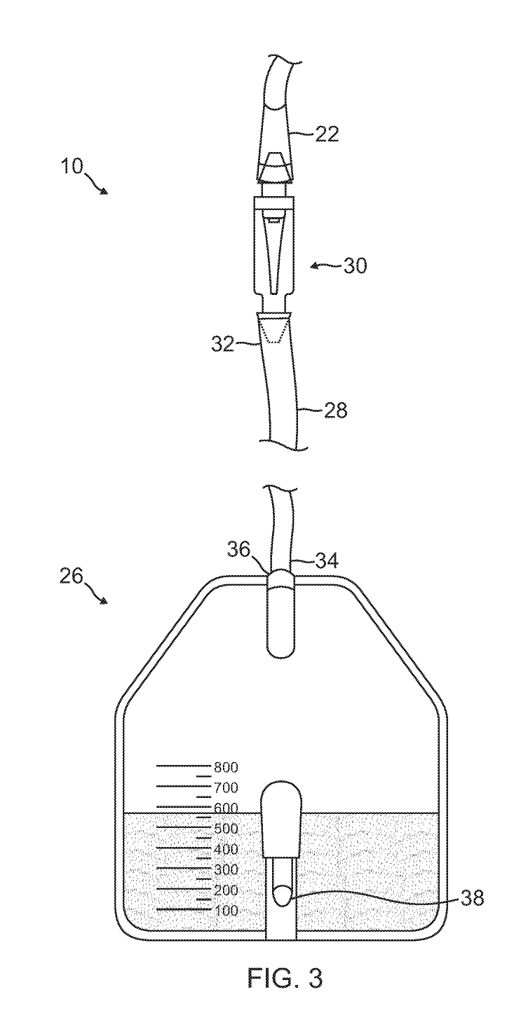

Catheterization allows the patient's urine to drain freely from the bladder for subsequent collection. As shown in FIG. 3, the Foley catheter 12 may be coupled to an external urine collection system, such as a drainage bag or container 26. The collection device 26 allows for the collecting and storing of urine for a given period of time so as to allow monitoring of urine flow, monitoring of the condition (e.g., appearance, consistency, etc.) of the urine, as well as permitting sampling of the collected urine for subsequent laboratory testing, in some cases, the catheter 12 may be coupled to the collection device 26 by way of tubing 28, for example.

As shown in FIG. 3, the drainage system 10 of the present disclosure further includes a valve assembly 30 positioned between the catheter 12 and the collection device 26 and providing a connection there between. As described in greater detail herein, the valve assembly 30 is configured to permit antegrade flow of urine from the catheter 12 into the collection device 26s while preventing retrograde flow (e.g. backflow) of urine from the collection device 26 into the catheter 12, thereby reducing the risk of CAUTI. For example, as shown, the valve assembly 30 has a first end directly coupled to the drainage port 22 of the catheter 12 and a second end direct coupled to a first end 32 of the tubing 28. The tubing 28 may include a second end 34 directly connected to an inlet post 36 of the collection device 26. Accordingly, the valve assembly 30 is configured to allow urine to flow from the catheter 12, through the tubing 28 and into the collection device 26. However, in the event that the collection device 28, tubing 28, and/or patient are manipulated and urine from the collection device 26 navels back towards the catheter 12, the valve assembly 30 is configured to prevent retrograde flow of urine therethrough and into the catheter 12, as described in greater detail herein.

It should be noted that the valve assembly 30 may be positioned between the catheter 12 and the collection device 28 in any arrangement, and need not be limited to the configuration shown in the figures. For example in some embodiments, the valve assembly 30 may be directly positioned between the second end 34 of the tubing 28 and the inlet port 36 of the collection device 26. In other embodiments, the valve assembly 30 may be directly positioned between the drain port 22 of the catheter 12 and the inlet portion 36 of the collection device 26, such that there is no tubing 28.

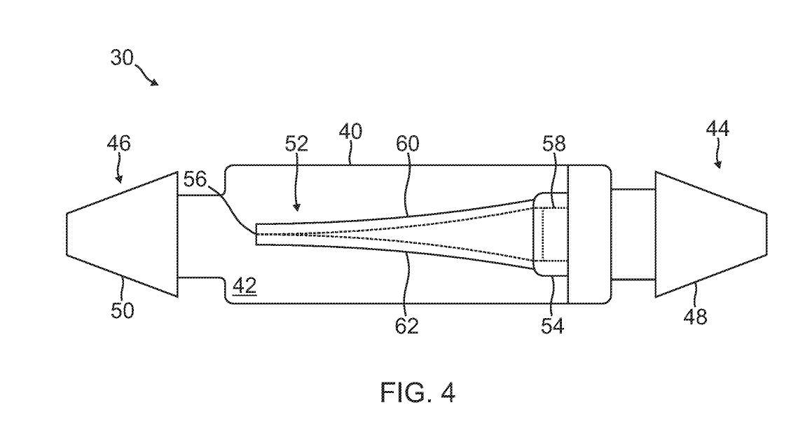

The valve assembly 30 of the present invention is a stand-alone device configured to be exchangeable and fit between most urinary catheters and collection bags and/or collection bag tubing. FIG. 4 is an enlarged side view of the valve assembly 30. As shown, the valve assembly 30 includes a housing 40 including a cavity/chamber within 42 and further includes an inlet end 44 and an outlet end 46. The inlet and outlet ends 44, 48 may each include a coupling element 48, 50 configured to provide a secure fit with the one of more portions of the catheter 12, tubing 28, and collection device 26, as shown in FIG. 3. For example, the coupling elements 48, 50 may generally be tapered and frustoconical in shape, such that coupling element 48 of the inlet end 44 may be securely connected (via press-fit) to the drain port 22 of the catheter 12 and coupling element 50 of the outlet end 48 may be securely connected (via press-fit) to the first end 32 of the tubing 34.

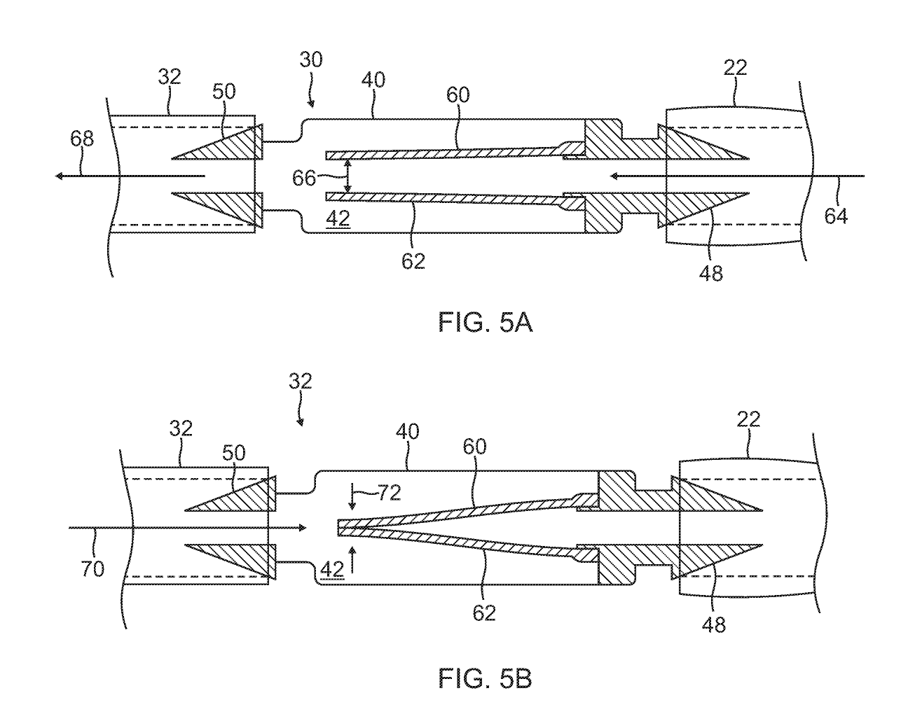

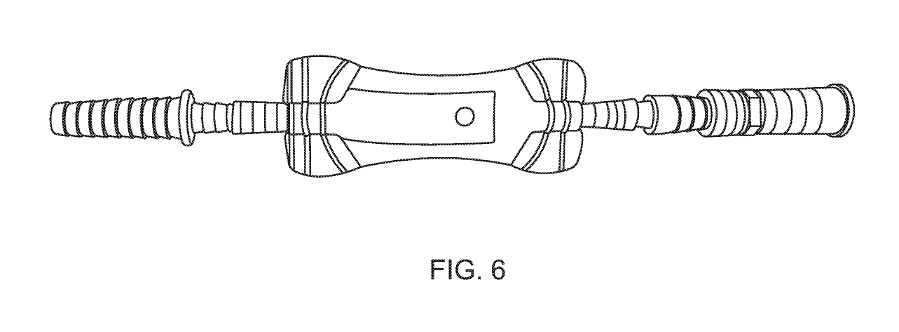

The valve assembly 30 further includes a valve 52 positioned within the cavity 42 of the housing 40. The valve 52 includes an open inlet 54 and an adjustable outlet 56 configured to move between a normally closed position, as shown, and an open position (shown in FIG. 5B). In some embodiments, the valve 52 may be a separate component, such that, when fully assembled, the open inlet 54 of the valve 52 is generally in engagement with the inlet end 44 of the housing 40. For example, the open inlet 54 of the valve 52 may be coupled to, and generally surround a nipple 58, or other connector, in a press-fit type fashion so as to provide a substantially leak-proof and/or airtight fit. Accordingly, upon draining of fluid (e.g., urine) from the catheter 12, the fluid may flow from the inlet end 44 of the housing 42 and through the open inlet 54 of the valve 52 info a fluid pathway formed, between the inlet and outlet 54, 58 of the valve 52.

As shown (illustrated in phantom), the valve 52 includes first and second elongated members 60, 82 joined along respective longitudinal edges thereof and sized to substantially span the valve inlet 54 and valve outlet 56. As described in greater detail herein, the first and second elongated members 60, 62 are configured to move so as to allow the outlet 56 of the valve 52 to adjust between open and closed positions. As such, the valve 52, including the first and second elongated members 60, 62, is constructed of an elastomeric material so as to allow sufficient flexibility and permit fluid to readily pass through the valve 52. For example, valve materials may include, but are not limited to, polyurethane, polyether-based polyurethane, polyvinyl chloride, polyesters, polyethylenes, polyamides, silicone and polyisoprenes, vinyl compounds, silicone rubber materials such as poly-dimethyl siloxane, thermoplastic polyester elastomers (TPEs), polyether block amides, and one or more combinations thereof. Additionally, or alternatively, the valve 52 material may have a substantially non-stick surface for permitting fluids and other materials to readily pass through the valve 52 and may include a coating so as to provide the same.

The housing 40 of the valve assembly 30 may be constructed of a transparent or translucent material, so as to provide a substantially unobstructed view of the valve 52 within. Accordingly, by providing a clear view into the interior cavity 42 of the housing 40, a clinician is able to ensure proper orientation of the valve direction and confirm that the valve assembly is positioned correctly (inlet end connected to catheter and outlet end connected to collection device), as well as allow a clinician to ascertain the working condition of the valve (e.g., clogging or malfunction of the valve).

FIG. 5A is a side view, partly in section, of the valve assembly 30 illustrating the valve 52 in an open position. As shown, upon receiving urine from the catheter, as indicated by arrow 64, the adjustable outlet 56 of the valve is configured to move from a closed position to an open position so as to allow urine to flow through completely through the valve 52. Accordingly, when in an open position, fluid is permitted to flow through the valve assembly 30 in an antegrade direction from the inlet end 44 and out of the outlet end 46 and to the collection device 26, as indicated by arrow 68.

As previously described, the first and second elongate members 60, 62 are configured to move relative to one another so as to transition between open and closed positions. In particular, the first and second elongate members 80, 62 are configured to separate from one another, as indicated by arrow 66, resulting in the outlet being in the open position upon receipt of urine flow from the catheter having a fluid pressure equal to or greater than a predefined threshold. In one embodiment, the predefined threshold is 3 cm H₂O pressure. However, in the event that the urine flow from the catheter is less the predefined threshold pressure, the fluid flow is insufficient in causing the first and second elongate members 60, 62 to separate from one another, and the outlet 56 of the valve 52 will remain in a normally closed position by default.

FIG. 5B is a side view, partly in section, of the valve assembly 30 illustrating the valve 52 in a normally closed position. As generally illustrated, when the adjustable outlet 56 is in a closed position, the first and second elongate members 60, 62 are generally in contact with one another at least at portions adjacent to the outlet 56, as indicated by arrows 72, wherein such contact is substantially leak-proof and/or airtight so as to prevent any fluid from entering the valve 52 from the outlet 56 end of the valve 52. Accordingly, when the outlet 56 is in a closed position, fluid is prevented from flowing through the valve assembly 30 in a retrograde direction from the outlet end 46 towards the inlet end 44 of the housing 40, thereby preventing backflow of urine info the catheter 12.

For example, upon fluid entering the outlet end 46 of the valve assembly 30, such as urine flowing: from the collection: device 26 (via tubing 28) towards the catheter 12, as indicated by arrow 70, the adjustable outlet 56 is configured to transition from the Open position to the closed position (indicated by arrows 72). When in a closed position, urine is prevented from flowing into the valve 52, and thus prevented from entering the catheter 12 and potentially causing subsequent infection.

Because the valve assembly 30 is a stand-alone device, it can be exchanged at any point during catheterization, which can be particularly beneficial in long-term catheterization. For example, over the course of a day, week, month, etc., multiple valve assemblies may be exchanged while maintaining the same catheter within the patient and/or collection device, thus saving time and costs.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. FIG. 6 shows a fully assembled catheter system with valve according to embodiments of the present invention, which those of skill in the art readily understand to tit between a patient and a drainage opening, as described above, and claimed below. Artisans shall readily understand that the instant system is able to be assembled from numerous off-of-the shelf components, and that those brands discussed, such as the FOLEY brand of catheters are meant to be illustrative not limiting.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein, it is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent, to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed:

1. A valve assembly for preventing retrograde flow of fluid in a drainage catheter, the valve assembly comprising:
a housing having an inlet end and an outlet end, the inlet end is configured to be couplable to a drainage catheter; and
a valve positioned within the housing and having an open inlet, an adjustable outlet, and a fluid pathway extending there between;
wherein the open inlet of the valve is configured to receive fluid from the drainage catheter and the adjustable outlet of the valve is configured to move between a closed position and an open position in response to receipt of fluid from the drainage catheter;
wherein, when the adjustable outlet is in an open position, fluid is permitted to flow through the valve assembly in an antegrade direction from the inlet end towards the outlet end of the housing, thereby permitting flow of fluid from the drainage catheter; and
wherein, when the adjustable outlet is in a closed position, fluid is prevented from flowing through the valve assembly in a retrograde direction from the outlet end towards the inlet end of the housing, thereby preventing backflow of fluid into the drainage catheter.

2. The valve assembly of claim 1, wherein the adjustable outlet of the valve is configured to move from the closed position to the open position in response to receipt of fluid flow from the drainage catheter having a fluid pressure equal to or greater than a predefined threshold.

3. The valve assembly of claim 2, wherein predefined threshold is 3 cmH₂O pressure.

4. The valve assembly of claim 2, wherein the adjustable outlet of the valve is configured to move from the open position to the closed position in response to a lack of fluid flow from the drainage catheter having a fluid pressure equal to or greater than a predefined threshold.

5. The valve assembly of claim t, wherein the adjustable outlet of the valve is configured to move from the open position to the closed position in response to receipt of fluid from the outlet end of the housing.

6. The valve assembly of claim 1, wherein the outlet end of the housing is configured to be coupled to a collection device configured to receive and collect fluid draining from the drainage catheter.

7. The valve assembly of claim 6, wherein the valve assembly is configured to prevent retrograde flow of fluid from the collection device into the drainage catheter,

8. The valve assembly of claim 1, wherein the valve comprises first and second elongated members joined along respective longitudinal edges thereof and steed to substantially span the valve inlet and valve outlet.

9. The valve assembly of claim 1, wherein the valve is constructed of an elastomeric material.

10. The valve assembly of claim 1, wherein the housing of the valve assembly is constructed of a transparent or translucent material configured to provide a view into an interior cavity of the housing.

11. A drainage catheter system for draining fluid from a patient, the system comprising:
a catheter comprising an elongate tubular body having a proximal end and a distal end, the proximal end having a drainage opening and the distal end having at least a drainage port in fluid communication with the drainage opening via a drainage lumen extending along a length of the body from the side drainage opening to the drainage port;
a collection device coupled to the catheter and configured to collect fluid received from the catheter; and
a valve assembly disposed between the catheter and the collection device and providing a connection tiers between, the valve assembly comprising:
a housing having an inlet end connected to the drainage port of the catheter and an outlet end connected to a portion of the collection device;
a valve positioned within the housing and having an open inlet, an adjustable outlet, and a fluid pathway extending there between;
wherein the open inlet of the valve is engaged with the housing inlet end and configured to receive fluid from the catheter and the adjustable outlet of the valve is configured to move between a normally closed position and an open position in response to receipt of fluid from the catheter;
wherein when the adjustable outlet is in an open position, fluid is permitted to flow through the valve assembly in an antegrade direction from the inlet end towards the outlet end of the housing, thereby permitting flow of fluid Iron- the catheter to the collection device, and, when the adjustable outlet is in a closed position, fluid is prevented from flowing through the valve assembly in a retrograde direction from the outlet end towards the inlet end of the housing, thereby preventing backflow of fluid from the collection device into the catheter.

12. The drainage catheter system of claim 11, wherein the adjustable outlet of the valve is configured to move from the closed position to the open position in response to receipt of fluid flow from the catheter having a fluid pressure equal to or greater than a predefined threshold.

13. The drainage catheter system of claim 12, wherein predefined threshold is 3 cmH₂O pressure.

14. The drainage catheter system of claim 12, wherein the adjustable outlet of the valve is configured to move from the open position to the closed position in response to a lack of fluid flow from the catheter having a fluid pressure equal to or greater than a predefined threshold.

15. The drainage catheter system of claim 11, wherein the adjustable outlet of the valve is configured to move from the open position to the closed position in response to receipt of fluid from the outlet end of the housing.

16. The drainage catheter system of claim 11, wherein the valve comprises first and second elongated members joined along respective longitudinal edges thereof and sized to substantially span the valve inlet and valve outlet.

17. The drainage catheter system of claim 11, wherein the valve is constructed of an elastomeric material.

18. The drainage catheter system of claim 11, wherein the housing of the valve assembly is constructed of a transparent or translucent material configured to provide a view into an interior cavity of the housing.

19. The drainage catheter system of claim 11, wherein the catheter is a urinary catheter configured to drain urine from a patient's bladder and the collection device is a urine collection bag.

20. The drainage catheter system of claim 11, wherein the valve assembly is discrete from the catheter and collection device.
